(19) United States
(12) Patent Application Publication
(10) Pub. No.: US 2025/0042839 A1
(43) Pub. Date: Feb. 6, 2025

(54) MULTIFUNCTIONAL N-OXIDE HYDROTROPES

(71) Applicant: NOURYON CHEMICALS INTERNATIONAL B.V., Amsterdam (NL)

(72) Inventors: Hanamanthsa Shankarsa BEVINAKATTI, Somerset, NJ (US); Sorel MURESAN, Stenungsund (SE); Crystal Sonya PRIESTER, Rahway, NJ (US); Kevin Michael SALMON, Nazareth, PA (US)

(73) Assignee: NOURYON CHEMICALS INTERNATIONAL B.V., Amsterdam (NL)

(21) Appl. No.: 18/783,530

(22) Filed: Jul. 25, 2024

Related U.S. Application Data
(60) Provisional application No. 63/515,686, filed on Jul. 26, 2023.

(51) Int. Cl. C07C 207/04; C07C 201/06; C08G 65/333; C11D 1/722; C11D 1/75; C11D 1/825

(52) U.S. Cl. CPC C07C 207/04; C07C 201/06; C08G 65/33382; C11D 1/8255; C11D 1/722; C11D 1/75

(57) ABSTRACT

A compound has the formula (I):

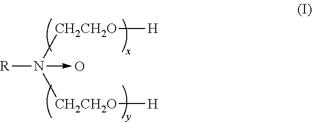

wherein R represents a fatty linear or branched, saturated or unsaturated alkyl group having 8-30 carbon atoms; and x+y is greater than about 5. This compound can be utilized in cleaning compositions.

### CROSS-REFERENCE TO RELATED APPLICATION

This application claims the benefit of U.S. Provisional Patent Application No. 63/515,686, filed Jul. 26, 2023, which is expressly incorporated herein by reference in its entirety.

### FIELD OF THE DISCLOSURE

The present disclosure relates to multifunctional hydrotropes and the use thereof in cleaning applications.

### BACKGROUND

As is well-known in the art, many surfactants are too hydrophobic to be soluble in water. Attempts to introduce such surfactants to water can result in cloudy or hazy solutions. In order to solubilize such surfactants, typically a hydrotrope must be added.

Hydrotropes in use include amphoteric surfactants and quaternary ammonium compounds as well as nonionic surfactants such as highly ethoxylated fatty acids and alkyl glucosides.

Quaternary ammonium compounds in use as hydrotropes include those, for example, described in U.S. Pat. No. 8,709,169 and European Patent No. 1 838 826. Compounds of this type are available from Nouryon (under the tradename BEROL®). However, quaternary ammonium salts are coming under increasing environmental pressure and products of this class require labelling.

It is an object of the present disclosure to develop a bio-based multifunctional hydrotrope with low or no environmental persistence and ecotoxicity for cleaning formulations. In addition, other objects, desirable features and characteristics will become apparent from the subsequent summary and detailed description, and the appended claims, taken in conjunction with the accompanying drawings and this background.

### BRIEF SUMMARY

This disclosure provides a compound of the formula (I):

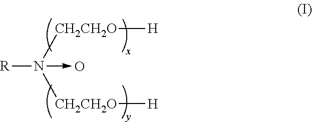

wherein
R represents a fatty linear or branched, saturated or unsaturated alkyl group having 8-30 carbon atoms; and
x+y is greater than about 5.

This disclosure also provides a compound of the formula (Ia):

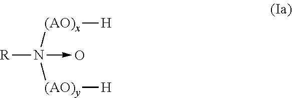

wherein
R represents a fatty linear or branched, saturated or unsaturated alkyl group having 8-30 carbon atoms;
each AO represents one or more alkylene oxide groups;
x+y is greater than about 5; and
the compound has a Griffin HLB value of from about 11 to about 17.

Surprisingly, it has been found that compounds of the formula (I) and (Ia) are very efficient hydrotropes and aid in the cleaning performance of compositions where they are present. In addition, such compounds are biodegradable and are expected to exhibit low/no toxicity.

### BRIEF DESCRIPTION OF THE DRAWINGS

The present disclosure will hereinafter be described in conjunction with the following drawing figures, wherein like numerals denote like elements, and

FIG. 1A is a photograph of the cleaning results described in the Examples relative to Samples 1 and 2 without NaOH;

FIG. 1B is also a photograph of the cleaning results described in the Examples relative to Samples 1 and 2 with NaOH;

FIG. 2A is a photograph of the cleaning results described in the Examples relative to Samples 11, 12, and 13 without NaOH;

FIG. 2B is also a photograph of the cleaning results described in the Examples relative to Samples 11, 12, and 13 with NaOH.

### DETAILED DESCRIPTION

The following detailed description is merely exemplary in nature and is not intended to limit any composition. Furthermore, there is no intention to be bound by any theory presented in the preceding background or the following detailed description.

Embodiments of the present disclosure are generally directed to compounds described above, compositions including the same, and methods for forming the same. For the sake of brevity, conventional techniques related to making such compounds and such compositions may not be described in detail herein. Moreover, the various tasks and process steps described herein may be incorporated into a more comprehensive procedure or process having additional steps or functionality not described in detail herein. In particular, various steps in the manufacture of such compounds and associated compositions are well-known and so, in the interest of brevity, many conventional steps will only be described briefly herein or will be omitted entirely without providing the well-known process details.

In this disclosure, the terminology “about” can describe values ±0.1, 0.5, 1, 2, 3, 4, 5, 6, 7, 8, 9, or 10%, in various embodiments. Moreover, it is contemplated that, in various non-limiting embodiments, it is to be appreciated that all numerical values as provided herein, save for the actual examples, are approximate values with endpoints or particular values intended to be read as “about” or “approximately” the value as recited.

The compounds and compositions disclosed herein may suitably comprise, consist of, or consist essentially of the components, elements, and process delineations described herein. The embodiments illustratively disclosed herein suitably may be practiced in the absence of any element which is not specifically disclosed herein.

This disclosure provides a compound of the formula (I):

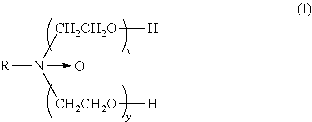

wherein R represents a fatty linear or branched, saturated or unsaturated alkyl group having 8-30 carbon atoms; and x+y is greater than about 5. Typically, the terminology “fatty” describes that the R group is derived from a fatty acid which is known in the art to be a carboxylic acid that includes a hydrocarbon chain and a terminal carboxyl group, especially any of those occurring as esters in fats and oils. This compound can also be described as, or used as, a hydrotrope.

In one embodiment, R is a linear alkyl group having 8-30 carbon atoms. In another embodiment, R is a branched alkyl group having 8-30 carbon atoms. Alternatively, R is a saturated alkyl group having 8-30 carbon atoms. Moreover, R can be an unsaturated alkyl group having 8-30 carbon atoms. R can be branched and saturated or branched and unsaturated. R can be linear and saturated or linear and unsaturated. In various embodiment, R has 8, 9, 10, 11, 12, 13, 14, 15, 16, 17, 18, 19, 20, 21, 22, 23, 24, 25, 26, 27, 28, 29, or 30 carbon atoms. Alternatively, R has 10 to 28, 12 to 24, 14 to 22, 16 to 20, or 16 to 18 carbon atoms. In various embodiments, R represents a cocoalkyl group. In various non-limiting embodiments, all values and ranges thereof, both whole and fractional, including and between those set forth above, are hereby expressly contemplated for use herein.

In other embodiments x+y is greater than about 5 and up to about 20, e.g. 6, 7, 8, 9, 10, 11, 12, 13, 14, 15, 16, 17, 18, 19, or 20. In various embodiments, x+y is from greater than about 5 and up to about 15, greater than about 5 and up to about 10, about 10 to about 15, greater than about 5 and up to about 20, about 10 to about 20, about 15 to about 20, etc. Alternatively, x+y may be described as greater than about 5 and up to about 20 or less, from about 10 and up to about 20 or less, or from about 15 and up to about 20 or less. Moreover, x+y may be described as from about 10 to about 20, about 11 to about 19, about 12 to about 18, about 13 to about 17, about 14 to about 16, etc. On average, x+y may be a whole or fractional number because of the polymeric nature of such groups. In other words, x+y may describe average values. In various non-limiting embodiments, all values and ranges thereof, both whole and fractional, including and between those set forth above, are hereby expressly contemplated for use herein.

In one embodiment, R represents a C8-C22 alkyl or alkenyl group and x+y is from about 5 to about 20. In another embodiment, R represents a C8-C22 alkyl or alkenyl group and x+y is from about 12 to about 17. In a further embodiment, R represents a C10-C18 alkyl or alkenyl group and x+y is from about 5 to about 20. In another embodiment, R represents a C10-C18 alkyl or alkenyl group and x+y is from about 12 to about 17. In another embodiment, R represents a fatty linear or branched, saturated or unsaturated alkyl group having 12 or 14 carbon atoms and x+y is from about 12 to about 17. In various embodiments, R represents a cocoalkyl group and x+y is from about 10 to about 20. Again, in various non-limiting embodiments, all values and ranges thereof, both whole and fractional, including and between those set forth above, are hereby expressly contemplated for use herein.

In still other embodiments, R represents a cocoalkyl group and x+y is greater than about 5 and up to about 20, e.g. 6, 7, 8, 9, 10, 11, 12, 13, 14, 15, 16, 17, 18, 19, or 20. In various embodiments, R represents a cocoalkyl group and x+y is from greater than about 5 and up to about 15, greater than about 5 and up to about 10, about 10 to about 15, greater than about 5 and up to about 20, about 10 to about 20, about 15 to about 20, etc. Alternatively, R represents a cocoalkyl group and x+y may be described as greater than about 5 and up to about 20 or less, from about 10 and up to about 20 or less, or from about 15 and up to about 20 or less. Moreover, R may represents a cocoalkyl group and x+y may be described as from about 10 to about 20, about 11 to about 19, about 12 to about 18, about 13 to about 17, about 14 to about 16, etc. On average, x+y may be a whole or fractional number because of the polymeric nature of such groups. In other words, x+y may describe average values. Again, in various non-limiting embodiments, all values and ranges thereof, both whole and fractional, including and between those set forth above, are hereby expressly contemplated for use herein.

This disclosure also provides a compound of the formula (Ia):

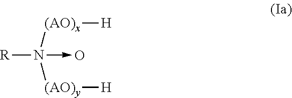

wherein R represents a fatty linear or branched, saturated or unsaturated alkyl group having 8-30 carbon atoms; each AO represents one or more alkylene oxide groups; x+y is greater than about 5; and the compound has a Griffin HLB value of from about 11 to about 17. In various non-limiting embodiments, all values and ranges thereof, both whole and fractional, including and between those set forth above, are hereby expressly contemplated for use herein. This compound can also be described as, or used as, a hydrotrope.

The terminology “AO” describes an alkyleneoxy group such as an ethyleneoxy or ethylene oxide (EO) group, a propyleneoxy or propylene oxide (PO) group, or a butyleneoxy or butylene oxide (BO) group. Any one or more of these groups can be utilized herein. Moreover, each of the AO groups described above can be independently any one or more of these groups. If more than one type of AO group is utilized, then the order may be any known in the art and may oriented in random or block orientations.

In one embodiment, one or more AO consists of ethylene oxide groups. In another embodiment, one or both AO consists of propylene oxide groups. In another embodiment, one or both AO consists of butylene oxide groups. In another embodiment, one or both AO consists of ethylene oxide groups and propylene oxide groups. In another embodiment, one or both AO consists of ethylene oxide groups and butylene oxide groups. In a further embodiment, one or both AO consists of propylene oxide and butylene oxide groups.

In the above, x+y is greater than about 5 or may be any value as is described above.

Moreover, the compound of Formula (Ia) has a Griffin HLB value of from about 11 to about 17, which is calculated using formulas well known in the art. In various embodiments, this value may be about 11.5 to about 16.5, about 12 to about 16, about 12.5 to about 15.5, about 13 to about 15, about 13.5 to about 14.5, or about 13.5 to about 14. In one embodiment, the Griffin HLB value is from about 15 to about 16. In various non-limiting embodiments, all values and ranges thereof, both whole and fractional, including and between those set forth above, are hereby expressly contemplated for use herein.

### Tertiary Amine:

In other embodiments, the aforementioned compound of Formula (I) is derived from a tertiary amine of the formula (II):

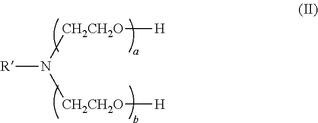

wherein R′ represents a fatty linear or branched, saturated or unsaturated alkyl group having 8-30 carbon atoms; and a+b is greater than about 5. In one embodiment, R′ represents a C₈-C₂₂ alkyl or alkenyl group and a+b is from about 5 to about 20. In another embodiment, R′ represents a C₈-C₂₂ alkyl or alkenyl group and a+b is from about 12 to about 17. In a further embodiment, R′ represents a C₁₀-C₁₈ alkyl or alkenyl group and a+b is from about 5 to about 20. In another embodiment, R′ represents a C₁₀-C₁₈ alkyl or alkenyl group and a+b is from about 12 to about 17. In this formula, a and/or b may independently be the same or different from x and/or y described herein. In various non-limiting embodiments, all values and ranges thereof, both whole and fractional, including and between those set forth above, are hereby expressly contemplated for use herein.

This disclosure also provides a process for preparing the compound of formula (I) wherein the method includes the step of oxidizing the tertiary amine of the formula (II) to give the compound of formula (I).

In various embodiments, a starting amine (Formula (II)) and an alkylene oxide (such as ethylene oxide) are charged to a reaction vessel. The alkylene oxide can be supplied to the reaction vessel randomly or in blocks. The amount of alkylene oxide necessary to obtain the desired degree of final product alkoxylation can be added all at once or sequentially over the time course of the reaction.

In one embodiment, the reaction vessel is initially charged with the total content of starting amine and a stoichiometric amount of alkylene oxide. Later, additional alkylene oxide is introduced in the amount necessary to achieve the ultimate desired degree of alkoxylation.

Typically, the reaction temperature is equal to or greater than 40° C., typically equal to or greater than 80° C., more typically equal to or greater than 100° C., more typically equal to or greater than 120° C. In various non-limiting embodiments, all values and ranges thereof, both whole and fractional, including and between those set forth above, are hereby expressly contemplated for use herein.

In another embodiment, the reaction temperature is kept between 100° C.-200° C. In various non-limiting embodiments, all values and ranges thereof, both whole and fractional, including and between those set forth above, are hereby expressly contemplated for use herein.

Pressure is typically monitored during the reaction so that the maximal pressure does not exceed 5 bar, and typically does not exceed 4.7 bar, most typically does not exceed 4.5 bar. In various non-limiting embodiments, all values and ranges thereof, both whole and fractional, including and between those set forth above, are hereby expressly contemplated for use herein.

Having obtained the alkoxylated amine of the formula (II), this compound can be converted to the final product of formula (I) or (Ia) by reaction with hydrogen peroxide. Methods for oxidizing amines with hydrogen peroxide are well-known, e.g. as described in U.S. Pat. No. 6,455,735 which is expressly incorporated herein in its entirety in various non-limiting embodiments.

In an embodiment, the alkoxylated amine of formula (II), hydrogen peroxide, and a chelating agent, for example, ethylenediaminetetraacetic acid (EDTA) or a salt thereof, for instance, the disodium salt, or, alternatively, one of the other chelating agents mentioned hereinbelow, are reacted in a reaction vessel at a temperature between 50° C.-100° C., most typically 55° C.-80° C. In various non-limiting embodiments, all values and ranges thereof, both whole and fractional, including and between those set forth above, are hereby expressly contemplated for use herein.

In one embodiment, the alkoxylated amine of formula (II) and the chelating agent are introduced to a reaction vessel along with water or other suitable solvent, and the temperature is manipulated to between 55° C.-65° C. Once the target temperature is reached, the hydrogen peroxide can be dosed into the reaction mixture and the temperature raised, for example, to around 70° C. If the solvent is water alone or in combination with a co-solvent, such as MPG (monopropylene glycol) or glycerol, there is no post-reaction work-up necessary and it is also not necessary to remove the solvent. On the other hand, it is also possible to carry out this reaction in lower alcohols, for example, methanol, ethanol, isopropyl alcohol etc., or in a mixture of such lower alcohols and water. However, these lower alcohol solvents, being volatile, are considered VOC and need to be removed. Other non-volatile solvents that can be used are other glycols such 1,3-propane diol, butane diols, diethylene glycols, dipropylene glycol etc., again, alone or in admixture with water. The use of water mixed with MPG is most typical, followed by the use of water mixed with glycerol. The amount of solvent is typically 0-80 wt % of the reaction mixture, most typically 40-60 wt %. In one typical embodiment, the solvent is removed from the product. In an embodiment, the solvent is not removed and is, therefore, present in the final product. In various non-limiting embodiments, all values and ranges thereof, both whole and fractional, including and between those set forth above, are hereby expressly contemplated for use herein.

Typically, catalysts are not needed, but the use of suitable catalysts is also contemplated.

In one embodiment, the reaction is carried out in a mixture of water+monopropylene glycol (MPG) as solvents. The solvents are not removed and the product once made is used “as is,” typically as a 50-60 wt % active in water+MPG. The reaction can also be carried out only in water or using water+glycerol. As the viscosity using water+glycerol is normally high, these are not the most typical unless they are diluted further to 40 wt % active. Instead of MPG, the reaction may also be carried out using other water miscible lower carbon chain length alcohols (which have VOC issues) or other diols or liquid polyols such as for example bio-based propane-1,3-diol, PEG or PPG. By using bio-based MPG or bio-based propane-1,3-diol, it is possible to achieve higher RCI (Renewable Carbon Content) of the product. The main RCI comes from the biobased secondary amine but it is possible to add RCI of the solvents also to the product. In a similar vein, it is also possible to increase the RCI by using Bio-EO to make the alkyl amine ethoxylate. The use of ethylene oxide made from bio-based ethanol can increase RCI to close to 90%. In various non-limiting embodiments, all values and ranges thereof, both whole and fractional, including and between those set forth above, are hereby expressly contemplated for use herein.

The active % of the product is typically 20-100%, most typically 40-60%. In various non-limiting embodiments, all values and ranges thereof, both whole and fractional, including and between those set forth above, are hereby expressly contemplated for use herein.

For example, the reaction may proceed as follows:

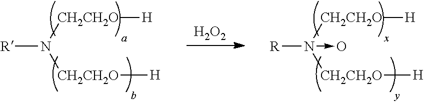

### Cleaning Composition:

This disclosure also provides an aqueous cleaning composition or formulation that includes compounds of this disclosure. Cleaning formulations including the compounds of this disclosure are useful for a variety of cleaning purposes. For example, such formulations can be formulated for household cleaning, industrial cleaning, all-purpose cleaning, car washing, acidic and caustic cleaning, deck and floor cleaning, hard surface cleaning, metal cleaning, food & beverage cleaning, automated and manual dishwash, laundry detergents and the like.

The aqueous cleaning composition of this disclosure includes (a) at least one compound of formula (I) or (Ia) as described above and (b) water. In various embodiments, the cleaning composition includes from about 0.1 to about 30, about 5 to about 25, about 10 to about 20, about 10 to about 15, about 0.1 to about 5, about 5 to about 15, or about 10 to about 30, weight percent actives of at least one compound of formula (I) and/or (Ia). In various non-limiting embodiments, all values and ranges thereof, both whole and fractional, including and between those set forth above, are hereby expressly contemplated for use herein.

In one embodiment, the aqueous cleaning composition also includes one or more surfactants. Alternatively, the composition may be free of such surfactants.

For example, the one or more surfactants may be one or more nonionic surfactants that may have formula (IV):

R³O—(PO)ₓ(EO)_{y}(PO)_{z}H (IV)

wherein R3 is a C8 to C18 linear or branched alkyl group, typically C8 to C12; PO is a propyleneoxy unit; EO is an ethyleneoxy unit; x=0-5, typically 0-4, and most typically 0-2; y=1-20, typically 1-12, more typically 2-8, and most typically 2-5; and z=0-5, typically 0-4, more typically 0-2, and most typically 0.

In various embodiments, and in addition to the 1-20 ethyleneoxy units, the C8-C18-alcohol alkoxylates may also include up to 5 propyleneoxy units. The number of propyleneoxy units, when present, may be as small as 0.1 mole PO per mole alcohol. The ethyleneoxy units and the propyleneoxy units may be added randomly or in blocks. The blocks may be added to the alcohol in any order. The alkoxylates may also include an alkyl group with 1-4 carbon atoms in the end position. Typically, the alkoxylates include 2-8 ethyleneoxy units and 0-2 propyleneoxy units. The alkyl group of the nonionic surfactants may be linear or branched, saturated or unsaturated. Suitable linear nonionic surfactants are C9-C11 alcohol+4, 5, 6, 7 or 8 moles of EO, C8-C10 alcohol+3, 4, 5, 6, 7 or 8 moles of EO, C12-C14 alcohol+3, 4, 5, 6, 7 or 8 moles of EO and C10-C14 alcohol+8 moles of EO+2 moles of PO. Suitable branched nonionic surfactants are 2-ethylhexanol+3, 4 or 5 moles of EO, 2-ethylhexanol+2 moles of PO+4, 5 or 6 moles of EO, 2-propylheptanol+3, 4, 5 or 6 moles of EO and 2-propylheptanol+1 mole of PO+4 moles of EO, C9 or C11 alcohol+4, 5, 6, 7 or 8 moles of EO, tridecyl alcohol+4, 5, 6, 7 or 8 moles of EO,. Another example is 2-butyloctanol+5, 6 or 7 moles of EO. Wherever the degree of alkoxylation is discussed, the numbers represent molar average numbers. In various non-limiting embodiments, all values and ranges thereof, both whole and fractional, including and between those set forth above, are hereby expressly contemplated for use herein.

In another embodiment, the one or more nonionic surfactants are chosen from nonionic alkylene oxide adducts, especially C8-C18 linear and branched alcohols (alcohol alkoxylates) and amine alkoxylates including 1-20 ethylencoxy units and 0-5 propyleneoxy units. The non-ionic alkyl polyglyceryl ethers made using C8-C18 linear and branched alcohol and 1-10 glycidol units or alkyl polyglycerylamines made using C8-C18 linear and branched alkyl amine and 1-10 glycidol units can also be used. In various non-limiting embodiments, all values and ranges thereof, both whole and fractional, including and between those set forth above, are hereby expressly contemplated for use herein.

In various embodiments, the amounts of the components may be as follows:
a) at least 0.05% by weight, typically at least 0.5% by weight, and at most 20% by weight, typically at most 15% by weight, and most typically at most 10% by weight, of alcohol alkoxylate,
b) at least 0.02% by weight, typically at least 0.1% by weight, and at most 20% by weight, typically at most 15% by weight, and most typically at most 10% by weight, of compound of formula (I) and/or (Ia), and
c) 0% by weight, typically at least 0.05% by weight, and at most 30% by weight, typically at most 20% by weight, more typically at most 15% by weight, and most typically at most 10% by weight, of alkali hydroxides, alkaline builders and/or alkaline complexing agents.

In various non-limiting embodiments, all values and ranges thereof, both whole and fractional, including and between those set forth above, are hereby expressly contemplated for use herein.

In various embodiments, the compositions include, or are free of, alkali hydroxides, alkaline builders and/or alkaline complexing agents.

The compositions may be acidic, neutral or alkaline. Alkaline compositions are typically based on alkali hydroxides, alkaline builders and/or complexing agents. The alkaline compositions are especially typical.

The alkali hydroxides that may be used typically are sodium or potassium hydroxide. The alkaline builders may be an alkali carbonate or an alkali hydrogen carbonate, such as sodium carbonate, potassium carbonate, sodium hydrogen carbonate or potassium hydrogen carbonate, an alkali salt of a silicate, such as sodium silicate or sodium metasilicate, or alkali salts of phosphates, such as sodium orthophosphate. Alkaline builders that act through complexation are, e.g. sodium pyrophosphate and sodium tripolyphosphate and the corresponding potassium salts. The builder/complexing agent may also be organic. Examples of organic builders/complexing agents are aminocarboxylates, such as Glutamic acid, N,N-diacetate (GLDA), Methylglycine, N,N-diacetate (MGDA), sodium nitrilotriacetate (Na3NTA), sodium ethylenediamine tetraacetate (EDTA), sodium diethylenetriamine pentaacetate, sodium 1,3-propylenediamine tetraacetate, and sodium hydroxyethylethylenediamine triacetate; aminopolyphosphonates, such as nitrilotrimethylene phosphonate; organic phosphates; polycarboxylates, such as citrates; and alkali salts of gluconic acid, such as sodium or potassium gluconates.

In neutral and acidic compositions, complexing and/or pH adjusting agents may also be added, such as citric acid, oxalic acid, acetic acid, sulfamic acid, hydrochloric acid.

In another embodiment, the cleaning composition includes one or more chelates.

In one embodiment, the chelate is at least one aminocarboxylate chelate chosen from methylglycinediacetic acid (MGDA), N,N-dicarboxymethyl glutamic acid (GLDA), N-hydroxyethyliminodiacetic acid, nitrilotriacetic acid (NTA), ethylenediaminetetraacetic acid (EDTA), N-hydroxyethyl-ethylenediaminetriacetic acid (HEDTA), diethylenetriaminepentaacetic acid (DTPA), ethylenediaminetetraproprionic acid triethylenetetraaminehexaacetic acid (TTHA), tetracetyl ethylene diamine (TAED), iminodisuccinic acid (IDS), ethanol diglycine (EDG), and the respective alkali metal, ammonium and substituted ammonium salts thereof. In a particularly typical embodiment, the aminocarboxylate chelate is chosen from EDTA, GLDA, MGDA, salts thereof, and combinations thereof.

In another embodiment, the chelate is a non-aminocarboxylate chelate including carboxylate functionality but not a nitrogen atom. In an embodiment, the non-aminocarboxylate chelate is a divalent or higher valency carboxylic acid. In an embodiment, the non-aminocarboxylate chelate is at least one member chosen from citric acid, isocitric acid, 2,3 hydroxycitric acid, tricarballylic acid, ethanetricarboxylic acid (HETA), aconitic acid, succinic acid, maleic acid, fumaric acid, oxaloacetic acid, ketoglutaric acid, butanetetracarboxylic acid, polycarboxylic acid, and the respective alkali metal, ammonium and substituted ammonium salts thereof. In a particularly typical embodiment, the non-aminocarboxylate chelate is chosen from citric acid and salts thereof.

When one or more chelates are present, they are typically present in a total combined chelate amount of greater than 0% by weight, typically at least 0.05% by weight, most typically at least 1% by weight, and at most 30% by weight, typically at most 20% by weight, more typically at most 15% by weight, and most typically at most 10% by weight. In various non-limiting embodiments, all values and ranges thereof, both whole and fractional, including and between those set forth above, are hereby expressly contemplated for use herein.

In an embodiment, in addition to one or more nonionic surfactants and/or chelates, the cleaning composition further includes, or is free of, one or more adjunct ingredients chosen from aesthetic agents, anti-filming agents, anti-redeposition agents, anti-spotting agents, anti-graying agents, beads, binders, biocides, bleach activators, bleach catalysts, bleach stabilizing systems, bleaching agents, brighteners, buffering agents, builders, carriers, clay, color speckles, control release agents, corrosion inhibitors, dish care agents, disinfectants, dispersant agents, draining promoting agents, drying agents, dyes, dye transfer inhibiting agents, enzymes, enzyme stabilizing systems, fillers, free radical inhibitors, fungicides, germicides, hydrotropes other than those of formula (I) and (Ia), opacifiers, perfumes, pH adjusting agents, pigments, processing aids, silicates, soil release agents, suds suppressors, anionic surfactants, cationic surfactants, stabilizers, thickeners, zeolite, and mixtures.

When one or more adjunct ingredients are present in the formulation, they are typically present in a total combined adjunct ingredient amount of greater than 0% by weight, typically at least 0.05% by weight, most typically at least 1% by weight, and at most 30% by weight, typically at most 20% by weight, more typically at most 15% by weight, and most typically at most 10% by weight. In various non-limiting embodiments, all values and ranges thereof, both whole and fractional, including and between those set forth above, are hereby expressly contemplated for use herein.

The present disclosure describes both diluted and concentrated compositions.

Diluted compositions of the present disclosure are typically clear and stable. The clarity interval suitably is between 0-40° C., typically between 0-50° C., and most typically between 0-60° C. This may be adapted by changing the ratio of hydrotrope to nonionic surfactant. The diluted compositions normally include at least 80% by weight of water, suitably at least 90% by weight, and normally at most 99.5% by weight of water, suitably at most 98% by weight. In various non-limiting embodiments, all values and ranges thereof, both whole and fractional, including and between those set forth above, are hereby expressly contemplated for use herein.

Concentrated compositions of the present disclosure are also typically clear and stable. The clarity interval suitably is between 0-40° C., typically between 0-50° C., and most typically between 0-60° C. This may be adapted by changing the ratio of hydrotrope to nonionic surfactant. The concentrate normally includes at least 50% by weight of water, suitably at least 70% by weight, and normally at most 95% by weight of water, suitably at most 90% by weight. In various non-limiting embodiments, all values and ranges thereof, both whole and fractional, including and between those set forth above, are hereby expressly contemplated for use herein.

There are several advantages connected with the use of the compounds of this disclosure as hydrotropes. First, they are excellent hydrotropes that also contribute to the cleaning performance of the compositions. Their cleaning efficiency is very good even at high dilutions of the compositions and they perform just as well as known components when used in compositions for cleaning hard surfaces. Yet, they are also readily biodegradable and are expected to have low/no toxicity and therefore are even further distinguished over the art.

In still other embodiments, this disclosure provides a method of cleaning an object to be cleaned including the step of contacting the object with the aqueous cleaning composition as described above. The step of contacting and/or cleaning may be any known in the art.

### EXAMPLES

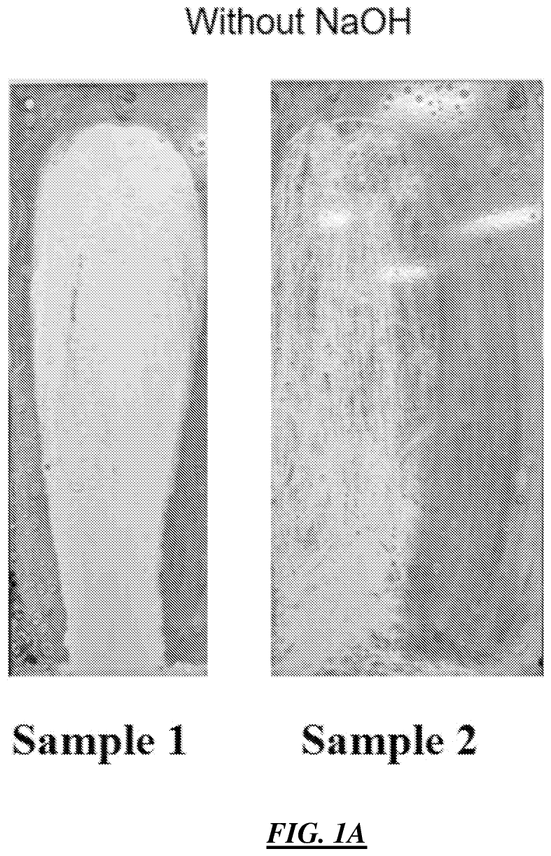

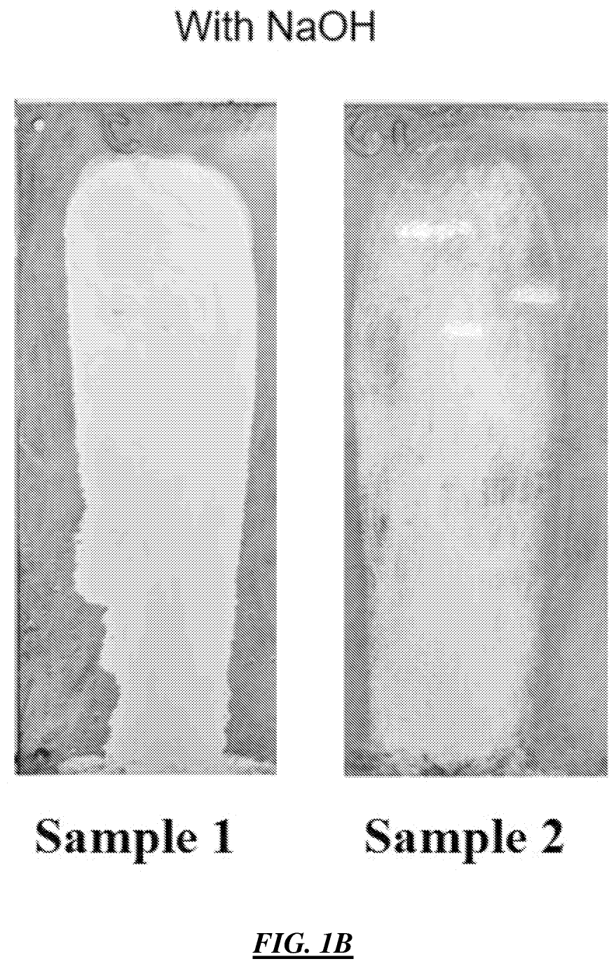

A series of samples were created as follows and evaluated to determine cloud point, cleaning performance, and appearance at room temperature (RT). The results are set forth in Table 1 below and shown in FIGS. 1A and 1B.

TABLE 1

| | | | no NaOH | | | | |
|---|---|---|---|---|---|---|---|
| | | | w/w of | Active % of | | | |
| | | Active | Hydrotrope | Hydrotrope | | | |
| Sample | | % of | in Formula | in Formula | Cloud | Cleaning | Appearance |
| # | Formulation | Hydrotrope | A | A | Point | Performance | @ RT |
| 1 | 5% Compound 1 + | 60% in | 4.00 | 2.40 | >70 | Good | Clear |
| | 4% Compound 2 | H₂O | | | | | |
| | (Comparative | | | | | | |
| | Hydrotrope) + | | | | | | |
| | 8% Compound 3 | | | | | | |
| 2 | Compound 4 | 65% | 3.69 | 2.40 | 54.5 | Good | Clear |
| | (Inventive | in H₂O | | | | | |
| | Hydrotrope) | | | | | | |
| | | | 2% NaOH | | | | |
| | | | w/w of | Active % of | | | |
| | | Active | Hydrotrope | Hydrotrope | | | |
| Sample | | % of | in Formula | in Formula | Cloud | Cleaning | Appearance |
| # | Formulation | Hydrotrope | B | B | Point | Performance | @ RT |
| 1 | 5% Compound 1 + | 60% in | 4.00 | 2.40 | 41.5 | Good | Clear |
| | 4% Compound 2 | H₂O | | | | | |
| | (Comparative | | | | | | |
| | Hydrotrope) + | | | | | | |
| | 8% Compound 3 | | | | | | |
| 2 | Compound 4 | 65% in | 3.69 | 2.40 | 36.7 | Good | Clear |
| | (Inventive | H₂O | | | | | |
| | Hydrotrope) | | | | | | |

Compound 1 is a mixture of alcohol ethoxylates.
Compound 2 is a Quaternary coco alkylamine ethoxylate.
Compound 3 is Glutamic acid, N,N-diacetic acid, tetrasodium salt.
Compound 4 is Cocoalkylamine ethoxylate N-oxide that includes 15 moles of ethylene oxide. This can be described as an inventive hydrotrope.

Formulation A is made without NaOH. Formula B is made using NaOH. Sample 1 is the benchmark using 4% of Compound 2 “as is.” When this value is corrected based on its active % (which is 60%), the result is 2.4%. Apart from the listed compounds above, the rest of Formulation A and B is water. The other Samples correspond to replacing Compound 2 with other hydrotropes. As the other hydrotropes are 65% active, less is used to keep the actual active % as 2.4% in the formulations.

This data shows that equivalent performance with the quaternary benchmark compound can be achieved by the compounds of this disclosure. This is superior to the benchmark because of the biodegradability of the instant compounds and is also unexpected.

Cloud Point can be determined using any method in the art, e.g. ASTM D2500, wherein a specimen is cooled at a specified rate and examined periodically. The temperature at which a cloud is first observed at the bottom of the test jar is recorded as the cloud point.

The cleaning performance was evaluated using the following non-mechanical cleaning tests. The formulations were applied on a painted steel panel soiled with engine grease, in a vertical position. After 30 seconds the panel was rinsed with tap water and the appearance at room temperature was evaluated visually by a human evaluator.

The results are set forth in FIGS. 1A and 1B. The appearance at room temperature was evaluated visually by a human evaluator.

Compound 4 was synthesized as follows. 60.48 g of cocoalkylamine 15EO is charged along with 18.68 g of water to a reaction vessel equipped with stirrers and maintained at a temperature of 45° C. Once the stirrers are started, 0.074 g of Glutamic acid, N,N-diacetic acid, tetrasodium salt (47% actives in H2O) are introduced to the reaction mixture.

When the water, ethoxylated amine, and Glutamic acid, N,N-diacetic acid, tetrasodium salt are charged, and the temperature of the reaction mixture is 45° C., 8.84 g of 35% active hydrogen peroxide are added dropwise keeping the temperature of the reaction mixture below 60° C. Once all the addition is over, the temperature is then increased to 75° C. and stirred for six hours.

Samples are taken from the reaction mixture for analysis and correction if needed. If the H2O2 content is too high, this is corrected with the addition of ethoxylated amine. If the free amine content is too high, this is corrected with the addition of H2O2. Samples should be taken at a three-hour intervals and corrections made at each sampling as necessary until no further corrections are needed.

The batch on pH can be adjusted as desired with H2SO4. The reaction mixture should continue to be stirred for 15 minutes after each addition.

Once a final sample of the reaction mixture has been taken, and the desired attributes of the product have been confirmed, the product mixture is cooled down to 30° C. and the temperature block turned off and the product can be discharged from the reactor into the desired packaging.

The same procedure as described above can be used to make similar compounds such as, but not limited to, 2EO, 5EO, 10 EO, and 20 EO N-oxides by using the respective ethoxylated amines.

Additional compounds were also attempted to be synthesized. For example, Cocoalkylamine ethoxylate N-oxide ethoxylated with 2 moles of ethylene oxide was synthesized at approximately 65 wt % actives in water but formed a gel in the reaction flask. Therefore, this product was not useable. An additional sample was synthesized at about 40 wt % actives in MPG and water (1:1). A further sample was synthesized at about 40 wt % actives in only water. Both of these samples were free flowing liquids. In addition, Cocoalkylamine ethoxylate N-oxide ethoxylated with 5 moles of ethylene oxide was synthesized at approximately 65 wt % actives in water but formed a gel after storage overnight.

An additional series of samples were created as follows and evaluated to determine cloud point, cleaning performance, and appearance at room temperature (RT). The results are set forth in Table 2 below. Sample 3 is a benchmark control sample. The cloud point, cleaning performance, and appearance are evaluated as described above.

TABLE 2

| | | | no NaOH | | | | |
|---|---|---|---|---|---|---|---|
| | | | w/w of | Active % of | | | |
| | | Active | Hydrotrope | Hydrotrope | | | |
| Sample | | % of | in Formula | in Formula | Cloud | Cleaning | Appearance |
| # | Formulation | Hydrotrope | A | A | Point | Performance | @ RT |
| 3 | 5% Compound 1 + | 100 | 2.40 | 2.40 | >70 | Good | Clear |
| | 2.4% Compound 5 | | | | | | |
| | (Comparative | | | | | | |
| | Hydrotrope) + | | | | | | |
| | 3.76% | | | | | | |
| | Compound 3 | | | | | | |
| 4 | 5% Compound 1 + | 65% in | 3.69 | 2.40 | 56.3 | Good | Clear |
| | 2.4% Compound 4 | H₂O | | | | | |
| | (Inventive | | | | | | |
| | Hydrotrope) + | | | | | | |
| | 3.76% | | | | | | |
| | Compound 3 | | | | | | |
| 5 | 5% Compound 1 + | 70% in | 3.43 | 2.40 | 59.6 | Good | Clear |
| | 2.4% Compound 4 | H₂O | | | | | |
| | (Inventive | | | | | | |
| | Hydrotrope) + | | | | | | |
| | 3.76% | | | | | | |
| | Compound 3 | | | | | | |
| | | | 2% NaOH | | | | |
| | | | w/w of | Active % of | | | |
| | | Active | Hydrotrope | Hydrotrope | | | |
| Sample | | % of | in Formula | in Formula | Cloud | Cleaning | Appearance |
| # | Formulation | Hydrotrope | B | B | Point | Performance | @ RT |
| 3 | 5% Compound 1 + | 100 | 2.40 | 2.40 | 63 | Good | Clear |
| | 2.4% Compound 5 | | | | | | |
| | (Comparative | | | | | | |
| | Hydrotrope) + | | | | | | |
| | 3.76% | | | | | | |
| | Compound 3 | | | | | | |
| 4 | 5% Compound 1 + | 65% in | 3.69 | 2.40 | 38 | Good | Clear |
| | 2.4% Compound 4 | H₂O | | | | | |
| | (Inventive | | | | | | |
| | Hydrotrope) + | | | | | | |
| | 3.76% | | | | | | |
| | Compound 3 | | | | | | |
| 5 | 5% Compound 1 + | 70% in | 3.43 | 2.40 | 44.2 | Good | Clear |
| | 2.4% Compound 4 | H₂O | | | | | |
| | (Inventive | | | | | | |
| | Hydrotrope) + | | | | | | |
| | 3.76% | | | | | | |
| | Compound 3 | | | | | | |

Compound 5 is a cocoalkyl amine ethoxylate methyl chloride quaternary compound.

The description of Formulas A and B is as set forth above.

This data shows that equivalent performance with the quaternary benchmark compound can be achieved by the compounds of this disclosure. This is superior to the benchmark because of the biodegradability and is also unexpected.

An additional series of samples were created as follows and evaluated to determine cloud point, cleaning performance, and appearance at room temperature (RT). The results are set forth in Table 3 below. Sample 6 is a benchmark control sample. The cloud point, cleaning performance, and appearance are evaluated as described above.

TABLE 3

| | | | no NaOH | | | | |
|---|---|---|---|---|---|---|---|
| | | | w/w of | Active % of | | | |
| | | Active | Hydrotrope | Hydrotrope | | | |
| Sample | | % of | in Formula | in Formula | Cloud | Cleaning | Appearance |
| # | Formulation | Hydrotrope | A | A | Point | Performance | @ RT |
| 6 | 5% Compound 1 + | 100% | 2.40 | 2.40 | >70 | Good | Clear |
| | 2.4% Compound 5 | | | | | | |
| | (Comparative | | | | | | |
| | Hydrotrope) + | | | | | | |
| | 3.76% | | | | | | |
| | Compound 3 | | | | | | |
| 7 | 5% Compound 1 + | 70% in | 3.43 | 2.40 | 59.1 | Good | Clear |
| | 2.4% Compound 4 | H₂O | | | | | |
| | (Inventive | | | | | | |
| | Hydrotrope) + | | | | | | |
| | 3.76% | | | | | | |
| | Compound 3 | | | | | | |
| 8 | 5% Compound 1 + | 65% in | 3.69 | 2.40 | 34.5 | Some | Slight Haze |
| | 2.4% Compound 6 | H₂O | | | | | |
| | (Comparative | | | | | | |
| | Hydrotrope) + | | | | | | |
| | 3.76% | | | | | | |
| | Compound 3 | | | | | | |
| 9 | 5% Compound 1 + | 40% in | 6.00 | 2.40 | N/A | N/A | Phase |
| | 2.4% Compound 7 | H₂O | | | | | Separates |
| | (Comparative | | | | | | |
| | Hydrotrope) + | | | | | | |
| | 3.76% | | | | | | |
| | Compound 3 | | | | | | |
| 10 | 5% Compound 1 + | 40% in | 6.00 | 2.40 | N/A | N/A | Phase |
| | 2.4% Compound 7 | MPG + H₂O | | | | | Separates |
| | (Comparative | | | | | | and Hazy |
| | Hydrotrope) + | | | | | | |
| | 3.76% | | | | | | |
| | Compound 3 | | | | | | |
| | | | 2% NaOH | | | | |
| | | | w/w of | Active % of | | | |
| | | Active | Hydrotrope | Hydrotrope | | | |
| Sample | | % of | in Formula | in Formula | Cloud | Cleaning | Appearance |
| # | Formulation | Hydrotrope | B | B | Point | Performance | @ RT |
| 6 | 5% Compound 1 + | 100% | 2.40 | 2.40 | 63 | Good | Clear |
| | 2.4% Compound 5 | | | | | | |
| | (Comparative | | | | | | |
| | Hydrotrope) + | | | | | | |
| | 3.76% | | | | | | |
| | Compound 3 | | | | | | |
| 7 | 5% Compound 1 + | 70% in | 3.43 | 2.40 | 42.7° C. | Good | Clear |
| | 2.4% Compound 4 | H₂O | | | | | |
| | (Inventive | | | | | | |
| | Hydrotrope) + | | | | | | |
| | 3.76% | | | | | | |
| | Compound 3 | | | | | | |
| 8 | 5% Compound 1 + | 65% in | 3.69 | 2.40 | 23.5 | Some | Hazy |
| | 2.4% Compound 6 | H₂O | | | | | |
| | (Comparative | | | | | | |
| | Hydrotrope) + | | | | | | |
| | 3.76% | | | | | | |
| | Compound 3 | | | | | | |
| 9 | 5% Compound 1 + | 40% in | 6.00 | 2.40 | N/A | N/A | Phase |
| | 2.4% Compound 7 | H₂O | | | | | separates |
| | (Comparative | | | | | | |
| | Hydrotrope) + | | | | | | |
| | 3.76% | | | | | | |
| | Compound 3 | | | | | | |
| 10 | 5% Compound 1 + | 40% in | 6.00 | 2.40 | N/A | N/A | Phase |
| | 2.4% Compound 7 | MPG + H₂O | | | | | separates |
| | (Comparative | | | | | | and Hazy |
| | Hydrotrope) + | | | | | | |
| | 3.76% | | | | | | |
| | Compound 3 | | | | | | |

The description of Formulas A and B is as set forth above.

Compound 6 is Cocoalkylamine ethoxylate N-oxide that is ethoxylated with 5 moles of ethylene oxide. This can be described as a comparative hydrotrope.

Compound 7 is Cocoalkylamine ethoxylate N-Oxide ethoxylated with 2 moles of ethylene oxide. This can be described as a comparative hydrotrope.

This data shows that equivalent performance with the quaternary benchmark compound can be achieved by the compounds of this disclosure. This is superior to the benchmark because of the biodegradability and is also unexpected. Moreover, this data shows that Cocoalkylamine ethoxylate N-oxide that is ethoxylated with 2 or 5 moles of ethylene oxide does not perform nearly as well as Compound 4 which is Cocoalkylamine ethoxylate N-oxide that includes 15 moles of ethylene oxide. This shows that the inventive compounds are superior to their lower ethoxylated counterparts which is unexpected.

A further series of samples were created as follows and evaluated to determine cloud point, cleaning performance, and appearance at room temperature (RT). The results are set forth in Table 4 below. The cloud point, cleaning performance, and appearance are evaluated as described above.

TABLE 4

| | | | No NaOH | | | | |
|---|---|---|---|---|---|---|---|
| | | | w/w of | Active % of | | | |
| | | Active | Hydrotrope | Hydrotrope | | | |
| Sample | | % of | in Formula | in Formula | Cloud | Cleaning | Appearance |
| # | Formulation | Hydrotrope | A | A | Point | Performance | @ RT |
| 11 | 5% Compound 1 + | 70% | 3.43 | 2.23 | 52.4 | Good | Clear |
| | 8% Compound 3 + | | | | | | |
| | 3.43% | | | | | | |
| | Compound 4 | | | | | | |
| | (Inventive | | | | | | |
| | Hydrotrope) | | | | | | |
| 12 | 5% Compound 1 + | 65% | 3.69 | 2.40 | 41.8 | Good | Clear |
| | 8% Compound 3 + | | | | | | |
| | 3.69% | | | | | | |
| | Compound 8 | | | | | | |
| | (Inventive | | | | | | |
| | Hydrotrope) | | | | | | |
| 13 | 5% Compound 1 + | 65% | 3.69 | 2.40 | 52.6 | Good | Clear |
| | 8% Compound 3 + | | | | | | |
| | 3.69% | | | | | | |
| | Compound 9 | | | | | | |
| | (Inventive | | | | | | |
| | Hydrotrope) | | | | | | |
| | | | 2% NaOH | | | | |
| | | | w/w of | Active % of | | | |
| | | Active | Hydrotrope | Hydrotrope | | | |
| Sample | | % of | in Formula | in Formula | Cloud | Cleaning | Appearance |
| # | Formulation | Hydrotrope | B | B | Point | Performance | @ RT |
| 11 | 5% Compound 1 + | 70% | 3.69 | 2.40 | 42.7 | Good | Clear |
| | 8% Compound 3 + | | | | | | |
| | 3.69% | | | | | | |
| | Compound 4 | | | | | | |
| | (Inventive | | | | | | |
| | Hydrotrope) | | | | | | |
| 12 | 5% Compound 1 + | 65% | 3.69 | 2.40 | Not | Some | Clear |
| | 8% Compound 3 + | | | | Evaluated | | |
| | 3.69% | | | | | | |
| | Compound 8 | | | | | | |
| | (Inventive | | | | | | |
| | Hydrotrope) | | | | | | |
| 13 | 5% Compound 1 + | 65% | 3.69 | 2.40 | Not | Some | Clear |
| | 8% Compound 3 + | | | | Evaluated | | |
| | 3.69% | | | | | | |
| | Compound 9 | | | | | | |
| | (Inventive | | | | | | |
| | Hydrotrope) | | | | | | |

The description of Formulas A and B is as set forth above.

Compound 1 is a mixture of alcohol ethoxylates.

Compound 3 is Glutamic acid, N,N-diacetic acid, tetrasodium salt.

Compound 4 is as described above and it Cocoalkylamine ethoxylate N-oxide that includes 15 moles of ethylene oxide. This can be described as an inventive hydrotrope.

Compound 9 is Cocoalkylamine ethoxylate N-oxide that includes 10 moles of ethylene oxide. This can be described as an inventive hydrotrope.

Compound 10 is Cocoalkylamine ethoxylate N-oxide that includes 20 moles of ethylene oxide. This can be described as an inventive hydrotrope.

The cleaning performance was evaluated using the aforementioned non-mechanical cleaning tests. The formulations were applied on a painted steel panel soiled with engine grease, in a vertical position. After 30 seconds the panel was rinsed with tap water and the appearance at room temperature was evaluated visually by a human evaluator.

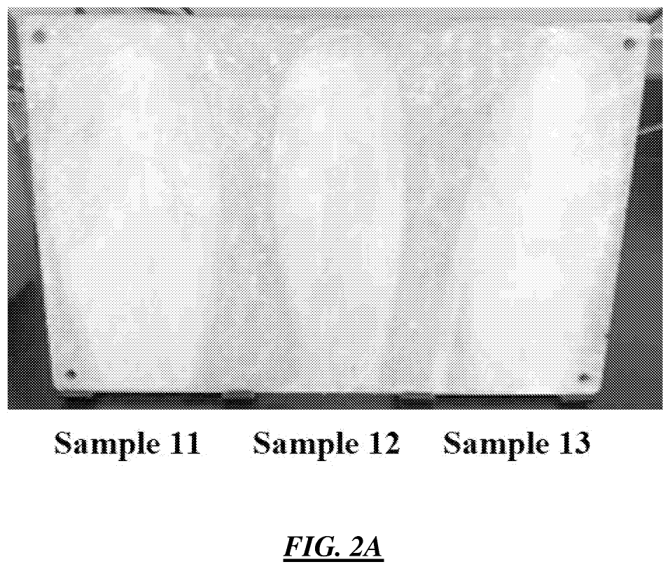

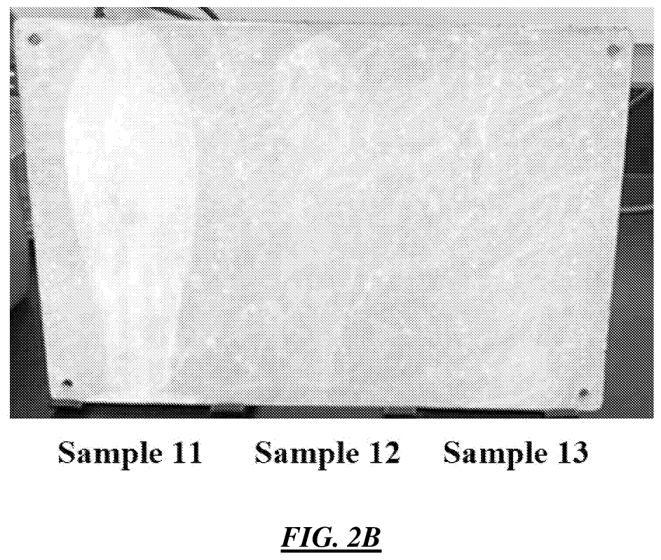

The results are set forth in FIGS. 2A and 2B. The appearance at room temperature was evaluated visually by a human evaluator.

This data shows that equivalent performance with the quaternary benchmark compound can be achieved by the compounds of this disclosure. This is superior to the benchmark because of the biodegradability and is also unexpected. Moreover, this data shows that Cocoalkylamine ethoxylate N-oxide that is ethoxylated with 2 or 5 moles of ethylene oxide does not perform nearly as well as Compounds 4, 8, and 9 which are Cocoalkylamine ethoxylate N-oxide that includes 15, 10, or 20 moles of ethylene oxide, respectively. Moreover, the aforementioned compounds also unexpectedly outperform ethoxylated counterparts wherein the ethoxylation is greater than 20 moles. This further shows that the inventive compounds are superior to their lower ethoxylated counterparts which is unexpected.

While the present disclosure has been described in conjunction with the specific embodiments set forth above, many alternatives, modifications and other variations thereof will be apparent to those of ordinary skill in the art. All such alternatives, modifications and variations are intended to fall within the spirit and scope of the present disclosure.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

What is claimed is:

1. A compound of the formula (I): 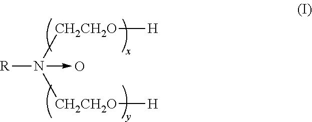
wherein
R represents a fatty linear or branched, saturated or unsaturated alkyl group having 8-30 carbon atoms; and
x+y is greater than about 5.

2. The compound of the formula (I) according to claim 1, wherein:
R represents a cocoalkyl group; and
x+y is from about 10 to about 20.

3. The compound of the formula (I) according to claim 1, wherein:
R represents a C₈-C₂₂ alkyl or alkenyl; and
x+y is from about 12 to about 17.

4. The compound of the formula (I) according to claim 1, wherein:
R represents a C₁₀-C₁₈ alkyl or alkenyl; and
x+y is from greater than about 5 and up to about 20.

5. The compound of the formula (I) according to claim 1, wherein:
R represents a C₁₀-C₁₈ alkyl or alkenyl; and
x+y is from about 12 to about 17.

6. The compound of the formula (I) according to claim 1, which derives from a tertiary amine of the formula (II): 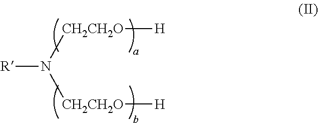
wherein
R′ represents a fatty linear or branched, saturated or unsaturated alkyl group having 8-30 carbon atoms; and
a+b is greater than about 5.

7. The compound of the formula (I) according to claim 6, wherein:
R′ represents a C₈-C₂₂ alkyl or alkenyl; and
a+b is from greater than about 5 and up to about 20.

8. The compound of the formula (I) according to claim 6, wherein:
R′ represents a C₈-C₂₂ alkyl or alkenyl; and
a+b is from about 12 to about 17.

9. The compound of the formula (I) according to claim 6, wherein:
R′ represents a C₁₀-C₁₈ alkyl or alkenyl; and
a+b is from greater than about 5 and up to about 20.

10. The compound of the formula (I) according to claim 6, wherein:
R′ represents a C₁₀-C₁₈ alkyl or alkenyl; and
a+b is from about 12 to about 17.

11. The compound of the formula (I) according to claim 1, wherein
R represents a fatty linear or branched, saturated or unsaturated alkyl group having 12 or 14 carbon atoms; and
x+y is from about 12 to about 17.

12. A process for preparing the compound of formula (I) according to claim 1, said method comprising the step of oxidizing a tertiary amine of the formula (II) to give the compound of formula (I): 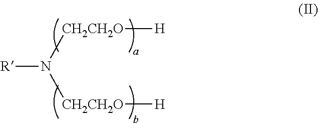
wherein
R′ represents a fatty linear or branched, saturated or unsaturated alkyl group having 8-30 carbon atoms; and
a+b is greater than about 5.

13. A compound of the formula (Ia): 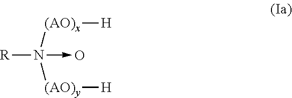
wherein
R represents a fatty linear or branched, saturated or unsaturated alkyl group having 8-30 carbon atoms;
each AO represents one or more alkylene oxide groups;
x+y is greater than about 5; and
said compound has a Griffin HLB value of from about 11 to about 17.

14. The compound of the formula (Ia) according to claim 13, wherein each AO consists of ethylene oxide groups.

15. The compound of the formula (Ia) according to claim 13, wherein each AO consists of ethylene oxide groups and propylene oxide groups.

16. The compound of the formula (Ia) according to claim 13 wherein the Griffin HLB value is from about 15 to about 16.

17. An aqueous cleaning composition comprising:
(a) at least one compound of formula (I) according to claim 1; and
(b) water.

18. The aqueous cleaning composition according to claim 17, which further comprises one or more nonionic surfactants.

19. The aqueous cleaning composition according to claim 18, wherein the more or more nonionic surfactants have formula (IV):
R³O—(PO)ₓ(EO)_{y}(PO)_{z}H (IV)
wherein
R³ is a C8 to C18 linear or branched alkyl group, typically C8 to C12;
PO is a propyleneoxy unit;
EO is an ethyleneoxy unit;
x=0-5, typically 0-4, and most typically 0-2;
y=1-20, typically 1-12, more typically 2-8, and most typically 2-5; and
z=0-5, typically 0-4, more typically 0-2, and most typically 0.

20. The aqueous cleaning composition according to claim 17, which further comprises one or more chelates.
